(19) United States
(12) Patent Application Publication
(10) Pub. No.: US 2025/0325270 A1
(43) Pub. Date: Oct. 23, 2025

(54) PULMONARY ARTERY FLOW RESTRICTOR

(71) Applicant: REGENTS OF THE UNIVERSITY OF MINNESOTA, Minneapolis, MN (US)

(72) Inventors: Juan Carlos Samayoa Escobar, Minneapolis, MN (US); Gurumurthy Hiremath Mallikarjun, Plymouth, MN (US); Shivangi Pandey, Blaine, MN (US); Aiden Golish, Minneapolis, MN (US); Rayan Haj Sakor, Minneapolis, MN (US); Calieb Prunty, St. Paul, MN (US); Travis Blohm, Minneapolis, MN (US)

(21) Appl. No.: 19/184,578

(22) Filed: Apr. 21, 2025

Related U.S. Application Data
(60) Provisional application No. 63/637,287, filed on Apr. 22, 2024.
(60) Provisional application No. 63/727,987, filed on Dec. 4, 2024.

(51) Int. Cl. A61B 17/12; A61B 17/00

(52) U.S. Cl. CPC A61B 17/12109; A61B 17/00234; A61B 17/12036; A61B 17/12122; A61B 17/12172; A61B 2017/00243; A61B 2017/00292; A61B 2017/00477; A61B 2017/00836; A61B 2017/00862; A61B 2017/00867; A61B 2017/00955; A61B 2017/12095

(57) ABSTRACT

The invention provides an endovascular device for use in restricting blood flow through the pulmonary artery. The device comprises a smart memory alloy wire frame partially covered with a thermoplastic layer. The device further comprises a screw mechanism coil coupled to the wire frame and/or thermoplastic layer and configured to allow delivery and retrieval of the device.

### CROSS-REFERENCE TO RELATED APPLICATION

This application claims priority to U.S. Provisional Application No. 63/637,287 that was filed on Apr. 22, 2024 and U.S. Provisional Application No. 63/727,987 that was filed on Dec. 4, 2024. The entire content of the applications referenced above is hereby incorporated by reference herein.

### BACKGROUND

A number of common congenital cardiac defects result in a left-to-right shunt, which allows oxygenated blood to flow from the left side of the heart to the right side, and back to the lungs. This can lead pulmonary over circulation and pulmonary arterial hypertension, causing rapid breathing, difficulty breathing, and/or low blood oxygen saturation. The current treatment for infants born with congenital heart disease with a significant left-to-right shunt is pulmonary surgical banding. Surgical pulmonary banding has also been applied in patients with congestive heart failure and in certain cases needing ventricular re-training. Although surgical pulmonary banding is a technically simple, it may be associated with high morbidity and mortality as it requires open surgery within the thoracic cavity.

At least over the last two decades, there has been slow advancement in the creation of a percutaneous implanted intraluminal medical device to minimize pulmonary artery flow. A currently available device, the Medtronic MVP™ Microvascular Plug (MVP), is designed for peripheral embolization rather than pulmonary artery flow restriction. Attempts have been made to modify the MVP for use as a flow restrictor; however, because it was not intended for this purpose, the MVP device is known to migrate off position and sometimes even collapse.

Therefore, there is a need for a stable device that will not migrate and that adequately restricts pulmonary flow without additional modifications.

### SUMMARY OF CERTAIN EMBODIMENTS OF THE INVENTION

The invention provides an endovascular device for use in restricting blood flow through the pulmonary artery. The device comprises a smart memory alloy (SMA) wire frame partially covered with a thermoplastic (e.g., polytetrafluoroethylene (PTFE)) layer. The device further comprises a screw mechanism coil coupled to the wire frame and/or thermoplastic layer and configured to allow delivery and retrieval of the device.

Accordingly, certain embodiments provide a pulmonary artery flow restrictor device comprising: SMA wire frame comprising a proximal end, a distal end, and at least one central aperture configured to allow blood flow through the device; a thermoplastic layer covering the proximal end of the SMA wire frame; and a screw mechanism coil proximally coupled to the thermoplastic layer, the screw mechanism coil configured to allow delivery and retrieval of the device.

In certain embodiments, the SMA wire is nitinol.

In certain embodiments, the thermoplastic layer is configured to direct blood flow through the at least one central aperture and prevent blood flow through the SMA wire frame.

In certain embodiments, the SMA frame is inward sloping toward the at least one central aperture.

In certain embodiments, the screw mechanism coil is coupled to the thermoplastic layer via a threaded cylindrical base extending proximally from the thermoplastic layer.

In certain embodiments, the screw mechanism coil is formed from SMA.

In certain embodiments, an outer diameter of the SMA wire frame is sized to exceed an internal diameter of a patient's pulmonary artery.

In certain embodiments, the SMA wire frame comprises a plurality of SMA wires, and the SMA wire frame comprises a plurality of connection points between adjacent SMA wires.

In certain embodiments, the connection points are laser welded.

In certain embodiments, the screw mechanism coil is configured to be reversibly coupled to a coil delivery wire.

In certain embodiments, the thermoplastic layer is polytetrafluoroethylene (PTFE).

In certain embodiments, the device further comprises at least one wire anchor coupled to an outer circumference of the SMA metal frame.

Certain embodiments provide a method of treating a patient with a congenital heart defect, the method comprising implanting the pulmonary artery flow restrictor device in the patient's pulmonary artery.

In certain embodiments, the congenital heart defect is a ventricular septal defect, and the device is implanted in the main pulmonary artery trunk.

In certain embodiments, the congenital heart defect is hypoplastic left heart syndrome, and the device is implanted in at least one of the left or right pulmonary artery branches.

In certain embodiments, the device is implanted using a cardiac catheter.

In certain embodiments, the device is configured to collapse within the catheter and to expand to a predetermined shape when deployed in the patient's pulmonary artery.

Other objects, features, and advantages of the present invention will be apparent to one of ordinary skill in the art from the following detailed description and drawings.

### BRIEF DESCRIPTION OF DRAWINGS

The present application can be understood by reference to the following drawings, wherein like reference numerals represent like elements. The drawings are merely exemplary to illustrate certain features that may be used singularly or in combination with other features and the present application should not be limited to the embodiments shown.

FIG. 1 is a perspective view of an embodiment of a pulmonary artery flow restrictor device in accordance with the present disclosure.

FIG. 2 is a top view of the distal end portion of an embodiment of the pulmonary artery flow restrictor device.

FIG. 3 is a side view of an embodiment of the pulmonary artery flow restrictor device, including a delivery coil.

FIG. 4 is a three-dimensional view of an embodiment of the pulmonary artery flow restrictor device, illustrating interconnections within the wire frame.

FIG. 5A is perspective view of the proximal end portion of an embodiment of the pulmonary artery flow restrictor device.

FIG. 5B is a side view of the pulmonary artery flow restrictor device shown in FIG. 5A.

FIG. 6 is a schematic drawing of an embodiment of the pulmonary artery flow restrictor device implanted in the main pulmonary artery in a subject with multiple ventricular septal defects.

FIG. 7 is a schematic drawing of an embodiment of the pulmonary artery flow restrictor device, including one device implanted in the left pulmonary artery branch and one device implanted in the left pulmonary artery branch, in a subject with hypoplastic left heart syndrome.

### DETAILED DESCRIPTION

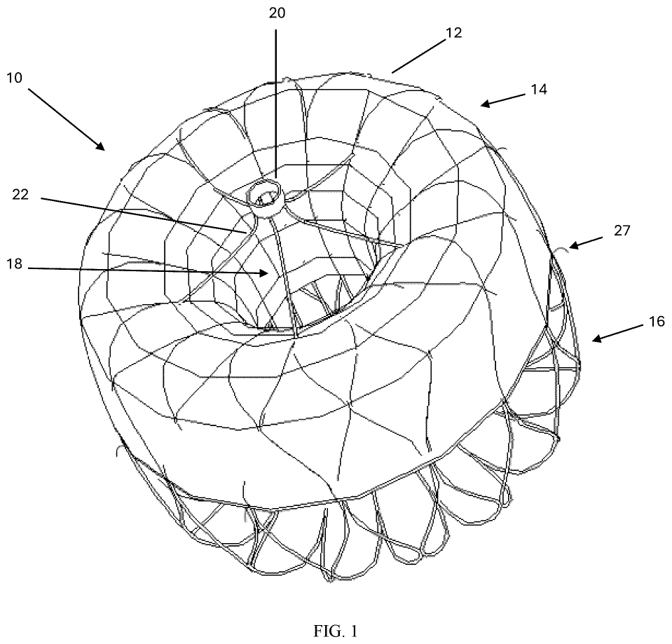

Referring now to the drawings wherein like reference numerals are used to identify like elements in the various views, FIG. 1 illustrates an exemplary embodiment of an endovascular pulmonary artery flow restrictor device 10. The device 10 is configured for placement inside a subject's pulmonary artery. While the device 10 can be used for both adult and pediatric patients, it offers a significant improvement in the treatment of newborns with congenital heart defects (CHDs). The device 10 can be used, for example, as temporary stabilizing solution for newborns awaiting more intensive treatment options. The device 10 is designed to prioritize safety, precision, and adaptability, as further described below.

The device 10 comprises a smart memory alloy (SMA) wire frame 12, including a proximal end portion 14 and a distal end portion 16. In an embodiment, the wire frame 12 is a nitinol wire mesh frame. In an embodiment, the wire frame 12 can have a wire thickness of about 0.05 mm to 0.15 mm. Due to the ability of the SMA to remember its shape and exhibit super elasticity, the device 10 can be compressed for insertion through a catheter, and then expand to its predetermined shape once deployed. This allows the device 10 to adapt to the pressures within the cardiovascular system without compromising its structural integrity or function.

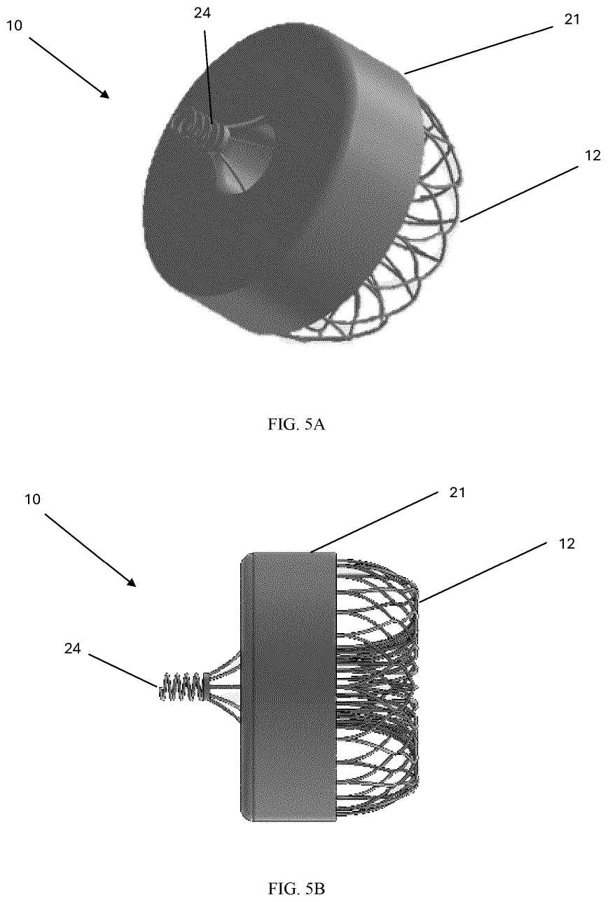

The device 10 includes at least one central aperture 18 configured to allow blood flow through the device. In an embodiment, the device 10 is modeled as a hollow cylinder, which allows blood to flow through it at a controlled rate. The proximal end 14 of the wire frame 12, can be covered by a thin thermoplastic layer 21, as shown and further described below with respect to FIGS. 5A and 5B.

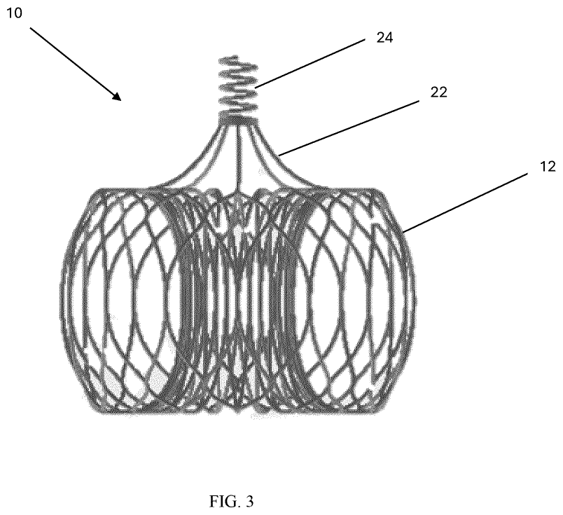

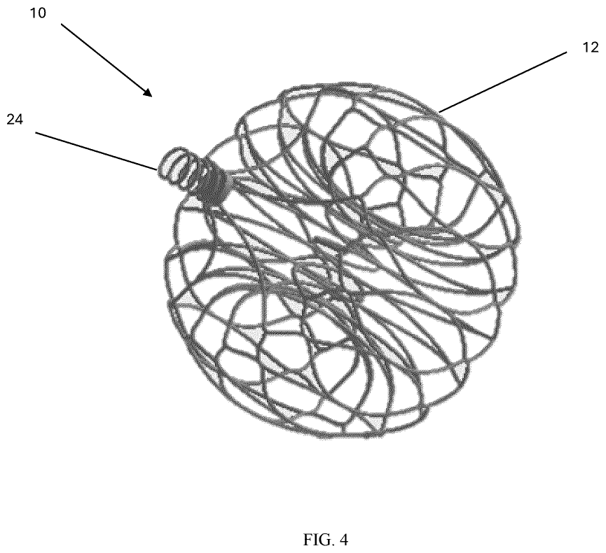

A cylindrical screw mechanism coil 24, shown to better advantage in FIGS. 3, 4, 5A, and 5B, is proximally coupled to a base 20 and the thermoplastic layer 21. The screw mechanism coil 24 is configured to allow delivery and retrieval of the device 10, such as via a catheter, as further described below. The screw mechanism coil 24 can be threaded to allow for attachment and detachment to the base 20. In an embodiment, the screw mechanism coil 24 is connected to the base 20 and the proximal end 14 of the wire frame 12 or thermoplastic layer 21 via a plurality of connecting nitinol wires 22. In the embodiment shown in FIG. 1, five evenly spaced supporting nitinol wires 22 connect the base 20 to the wire frame 12/thermoplastic layer 21. The screw mechanism coil 24 can be made of the same material as the wire frame 12 (e.g., a SMA, such as nitinol). Together, the screw mechanism coil 24 and base 20 form the delivery and retrieval component of the device 10.

The pulmonary artery flow restrictor device 10 is further described according to its key features below:

### Preventing Migration

The device 10 has a focus on anti-migration geometry. This requirement is achieved by slightly oversizing it in comparison to the target blood vessel (e.g., the pulmonary artery trunk or the right and left pulmonary artery branches). This ensures that the device 10 remains securely in place, resisting the natural forces exerted by blood circulation and the artery itself pulsating.

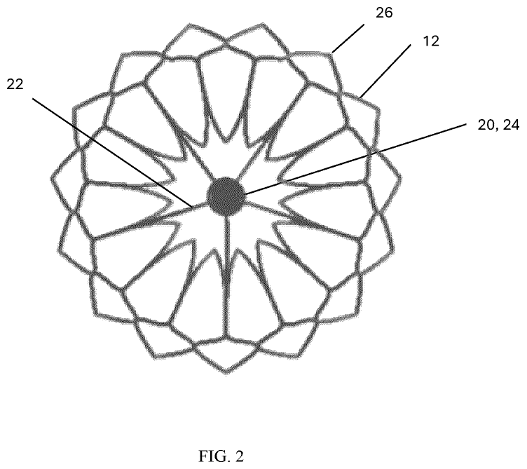

To address the issue of post-placement migration, the device 10 uses radial oversizing as the preventive mechanism and also includes one or more soft, curved outer anchors 27, as shown in FIG. 1. In the embodiment shown in FIG. 2, the outer radius of the distal end portion 16 of the wire frame 12 comprises fifteen contact points 26 which maintain contact with the artery wall when the device 10 is implanted; in other embodiments, however, the device 10 can include fewer or more than fifteen contact points 26. When implanted within the artery, the radial force of the wire frame 12 is exerted on the artery wall, and by extension, the wall's counterforce provides enough force to hold the device 10 in place. A large radial force increases the static frictional force between the wall and the device 10, allowing it to withstand the pressure and drag force exerted by the flow of blood. In addition, the anchors 27 embedded within the artery wall help to secure the device 10 in place.

In an embodiment, the device 10 can be oversized to exceed the pulmonary artery's internal diameter by approximately 20% or greater, ensuring a secure fit that mitigates the risk of displacement in the direction of flow. An average pulmonary artery inner diameter for an infant is about 6 mm; therefore, an outer diameter of the device, when fully expanded, can be about 8 mm, for example. This deliberate oversizing can significantly enhance the device's stability and reliability within the pulmonary artery. Due to the inherent diversity in patient anatomy, especially in the pediatric demographic with congenital heart defects, the device 10 can include a spectrum of device sizes to elevate the treatment efficacy and ensure that each device placement is appropriate for the individual's anatomy and therapeutic needs.

### Flow Restriction

The SMA wire frame 12 can be enveloped in a thin thermoplastic layer 21, as described above, allowing the device 10 to achieve blood flow restriction. The layering of these components is configured to ensure that blood flow is directed primarily through the central aperture 18, and that blood flow is prevented from flowing through the wire frame 12. In an embodiment, the central aperture 18 can be about 2.5 mm in diameter; however, other smaller and larger diameters are contemplated to accommodate diverse patient anatomy.

The device 10 is configured to be placed within the pulmonary artery so that the central aperture 18 is concentric with the blood vessel, minimizing any potential for turbulence. The thermoplastic layer 21 covering the proximal portion of the device is configured to mitigate blood flow disturbances in the pulmonary artery branches leading into the lungs. An inward-sloping design of the wire frame 12 surrounding the central aperture 18 also helps to guides blood flow toward the central aperture 18, facilitating a smoother transition of blood flow and significantly reducing the likelihood of turbulence and blood recirculation.

### Frame Design

As discussed above, the SMA wire frame 12 can be made of nitinol, which provides advantageous shape memory properties, flexibility, and biocompatibility. In addition to the frame's contact points 26 with the artery wall, and the interlocked delivery system including the base 20 and screw mechanism coil 24, the frame can include a plurality of connection points between adjacent nitinol wires that form the frame 12. For example, the frame 12 can include six or more connection points between adjacent nitinol wires on the outside diameter of the device 10. The connection points can be laser welded to provide structural support for the device. In an embodiment, the inner diameter of the frame 12 has no connections, allowing the geometry to be compressed to a very small diameter for device delivery (e.g., via a 4-French catheter).

### Deliverability/Retrieval System

The device 10 is configured to be used with a delivery and retrieval system including a specialized coil delivery wire (not shown). In an embodiment, the coil delivery wire comprises a 0.035-inch wire portion (e.g., a nitinol wire) with a coil portion at one end. The coil portion of the coil delivery wire is designed to be threaded together with the screw mechanism coil 24 of the device 10. For example, the coil portion of the coil delivery wire can be turned clockwise to engage with the screw mechanism coil 24 for loading of the device 10. Similarly, the coil portion of the coil delivery wire can be turned counterclockwise to disengage with the screw mechanism coil 24 once the device 10 is in place. Retrieval of the device 10 can be performed in the same manner using the coil delivery wire.

The wire portion of the coil delivery wire can have an extended length that is intentionally crafted to facilitate manipulation and removal in small children and adult patients. The coil delivery wire can function as a guidewire within a catheter (e.g., a 4-French catheter) that has been introduced through a minimally invasive incision near the patient's groin area into the femoral vein, navigated through the vascular system, and advanced into the pulmonary artery under the guidance of fluoroscopic imaging. Once the device 10 and the coil delivery wire have been threaded via the screw mechanism coil 24, the device is inserted and gradually collapses into the catheter. This process facilitates a smooth transition of the device 10 into the catheter as is pushed into position. Once the device 10 has been implanted in the pulmonary artery, the coil portion of the coil delivery wire can be disengaged from the screw mechanism coil 24, and the coil delivery wire can be retracted through the catheter.

### Use

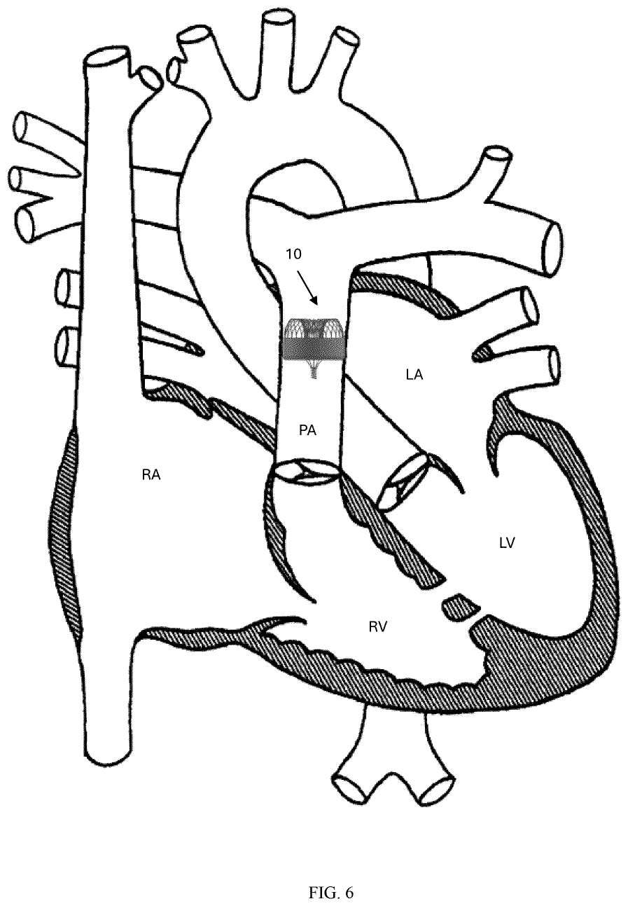

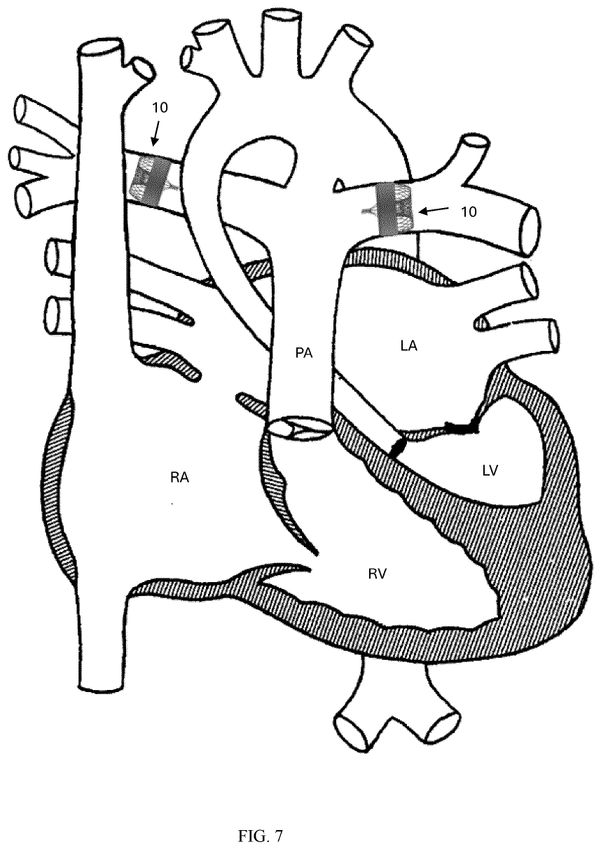

This device 10 is designed for the treatment of specific CHDs in infants and newborns. For example, the device 10 can be used for the treatment of multiple ventricular septal defects. In this case, one device 10 can be implanted in the main pulmonary artery trunk, as shown in FIG. 6. In another example, the device 10 can be used for the treatment of Hypoplastic Left Heart Syndrome. In this case, two devices 10 can be used-one implanted in the proximal left pulmonary artery branch and one implanted in the proximal right pulmonary artery branch-as shown in FIG. 7. The device 10 can serve as a temporary solution (e.g., the device can remain implanted in the heart for a period of about 4-6 months) until the patient reaches an age where more intensive treatment options can be safely administered.

### Device Placement

In pediatric patients, the device 10 can be placed using a 4-French catheter, which is recognized for its slender profile and compatibility in this patient population. The catheter can be introduced through a minimally invasive incision near the patient's groin area into the femoral vein, then navigated through the vascular system and advanced toward the heart under the guidance of fluoroscopic imaging. This imaging technique provides the surgeon with a real-time view of the device's location in the pulmonary artery, ensuring accurate placement. Once the device 10 reaches the targeted location within the pulmonary artery, it is deployed from the catheter until the entire length of the device 10 and coil delivery wire exit the distal end of the catheter. If the device 10 position is unsatisfactory, the surgeon can pull the screw mechanism coil 24 and device 10 back into the catheter. When the desired coil position is obtained, the catheter is kept in place and the delivery wire is turned counter-clockwise to detach the coil delivery wire from the screw mechanism coil 24.

### Device Removal

The device 10 can be removed by a surgeon employing a standard gooseneck snare designed to retrieve and manipulate foreign objects in the body. The snare can be introduced through a 4-French catheter using a similar intravenous path as the initial placement.

Fluoroscopic imaging can again be utilized to navigate the snare to the endovascular device 10. The snare's design allows it to securely latch onto the device at the screw mechanism coil 24 connection point. The snare is tightened around the device, which is then retracted into the catheter, is its collapsed state.

Various embodiments are described herein to various apparatuses, systems, and/or methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” or “an embodiment,” or the like, means that a particular feature, structure, or characteristic described in connection with the embodiment is included in at least one embodiment. Thus, appearances of the phrases “in various embodiments,” “in some embodiments,” “in one embodiment,” or “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation given that such combination is not illogical or non-functional.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

The terms “about” and “approximately” may be used throughout the specification when referring to a measurable value, such as an amount, a distance, a temporal duration, and the like. The terms “about” and “approximately” are meant to encompass variations of ±20% or ±10%, in certain embodiments ±5%, in certain embodiments ±1%, in certain embodiments ±0.1% from the specified value, as such variations are appropriate in accordance with the present disclosure.

Any patent, publication, or other disclosure material, in whole or in part, which is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A pulmonary artery flow restrictor device comprising:
a smart memory alloy (SMA) wire frame comprising a proximal end, a distal end, and at least one central aperture configured to allow blood flow through the device;
a thermoplastic layer covering the proximal end of the SMA wire frame; and
a screw mechanism coil proximally coupled to the thermoplastic layer, the screw mechanism coil configured to allow delivery and retrieval of the device.

2. The device of claim 1, wherein the SMA wire is nitinol.

3. The device of claim 1, wherein the thermoplastic layer is configured to direct blood flow through the at least one central aperture and prevent blood flow through the SMA wire frame.

4. The device of claim 1, wherein the SMA frame is inward sloping toward the at least one central aperture.

5. The device of claim 1, wherein the screw mechanism coil is coupled to the thermoplastic layer via a threaded cylindrical base extending proximally from the thermoplastic layer.

6. The device of claim 1, wherein the screw mechanism coil is formed from SMA.

7. The device of claim 1, wherein an outer diameter of the SMA wire frame is sized to exceed an internal diameter of a patient's pulmonary artery.

8. The device of claim 1, wherein the SMA wire frame comprises a plurality of SMA wires, and wherein the SMA wire frame comprises a plurality of connection points between adjacent SMA wires.

9. The device of claim 8, wherein the connection points are laser welded.

10. The device of claim 1, wherein the screw mechanism coil is configured to be reversibly coupled to a coil delivery wire.

11. The device of claim 1, wherein the thermoplastic layer is polytetrafluoroethylene (PTFE).

12. The device of claim 1, further comprising at least one wire anchor coupled to an outer circumference of the SMA metal frame.

13. A method of treating a patient with a congenital heart defect, the method comprising implanting the device of claim 1 in the patient's pulmonary artery.

14. The method of claim 13, wherein the congenital heart defect is a ventricular septal defect, and wherein the device is implanted in the main pulmonary artery trunk.

15. The method of claim 13, wherein the congenital heart defect is hypoplastic left heart syndrome, and wherein the device is implanted in at least one of the proximal left or right pulmonary artery branches.

16. The method of claim 13, wherein the device is implanted using a cardiac catheter.

17. The method of 16, wherein the device is configured to collapse within the catheter and to expand to a predetermined shape when deployed in the patient's pulmonary artery.
